(19) United States
(12) Patent Application Publication
(10) Pub. No.: US 2026/0025604 A1
(43) Pub. Date: Jan. 22, 2026

(54) DETECTION DEVICE

(71) Applicant: Japan Display Inc., Tokyo (JP)

(72) Inventors: Keiichi SAITO, Tokyo (JP); Atsunori OYAMA, Tokyo (JP); Gen KOIDE, Tokyo (JP)

(21) Appl. No.: 19/341,252

(22) Filed: Sep. 26, 2025

Related U.S. Application Data
(63) Continuation of application No. PCT/JP2024/012525, filed on Mar. 27, 2024.

(30) Foreign Application Priority Data
Mar. 30, 2023 (JP) 2023-054751

(51) Int. Cl. H04N 25/78; G06V 40/13; H10K 30/60

(52) U.S. Cl. CPC H04N 25/78; G06V 40/1318; H10K 30/60

(57) ABSTRACT

According to an aspect, a detection device includes: a photodiode; and a detection circuit that is coupled to an anode or a cathode of the photodiode via a switch and is configured to output a sensor value corresponding to a current output from the photodiode. The detection circuit is configured to measure the current output from the photodiode and output the sensor value, after a lapse of a predetermined period from a first time point at which the switch is turned on.

### CROSS-REFERENCE TO RELATED APPLICATION

This application claims the benefit of priority from Japanese Patent Application No. 2023-054751 filed on Mar. 30, 2023 and International Patent Application No. PCT/JP2024/012525 filed on Mar. 27, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (for example, Japanese Patent Application Laid-open Publication No. 2009-032005). Such optical sensors each include a plurality of photodiodes each including an organic semiconductor material as an active layer. As described in International Patent Application Publication No. WO/2020/188959, each of the photodiodes is located between a lower electrode and an upper electrode; and, for example, the lower electrode, an electron transport layer, the active layer, a hole transport layer, and the upper electrode are stacked in this order.

Optical sensors using organic photodiodes (OPDs) are required to improve the detection accuracy.

For the foregoing reasons, there is a need for a detection device capable of improving the detection accuracy.

### SUMMARY

According to an aspect, a detection device includes: a photodiode; and a detection circuit that is coupled to an anode or a cathode of the photodiode via a switch and is configured to output a sensor value corresponding to a current output from the photodiode. The detection circuit is configured to measure the current output from the photodiode and output the sensor value, after a lapse of a predetermined period from a first time point at which the switch is turned on.

### BRIEF DESCRIPTION OF THE DRAWINGS

FIG. 1 is a plan view schematically illustrating a detection device according to an embodiment;

FIG. 2 is a sectional view along II-II′ of FIG. 1;

FIG. 3 is a circuit diagram illustrating a configuration example of a control circuit according to the embodiment;

FIG. 4 is a timing waveform diagram illustrating an operation example of the detection device according to the embodiment; and

FIG. 5 is a flowchart illustrating an exemplary method for setting the number of times of measurement of a detection circuit of the detection device according to the embodiment.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

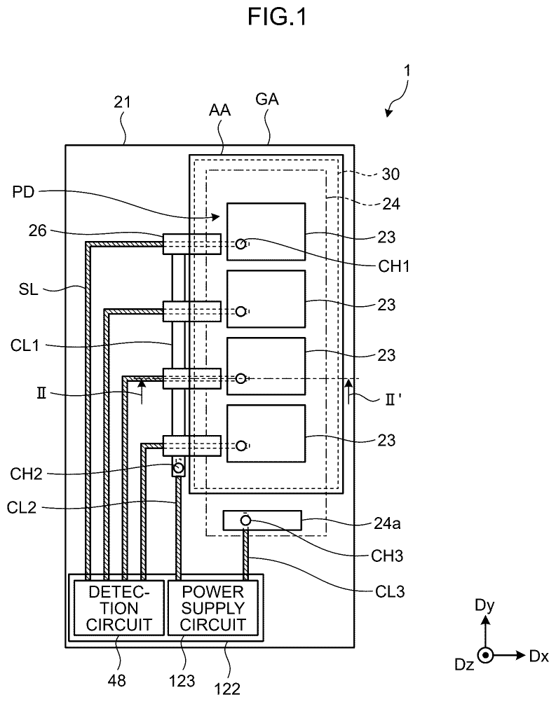

FIG. 1 is a plan view schematically illustrating a detection device according to an embodiment. As illustrated in FIG. 1, a detection device 1 includes a substrate 21, a plurality of photodiodes PD, a plurality of signal lines SL, a plurality of shield layers 26, power supply wiring lines CL1, CL2, and CL3, and a control circuit 122.

The substrate 21 has a detection area AA and a peripheral area GA. The detection area AA is an area provided with the photodiodes PD. The peripheral area GA is an area between the outer perimeter of the detection area AA and the ends of the substrate 21 and is an area not provided with the photodiodes PD. The signal lines SL and the control circuit 122 are provided in the peripheral area GA of the substrate 21.

In the following description, a first direction Dx is one direction in a plane parallel to the substrate 21. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term “plan view” refers to a positional relation when viewed in a direction orthogonal to the substrate 21.

The detection device 1 includes the photodiodes PD as optical sensor elements. Each of the photodiodes PD outputs an electrical signal corresponding to light emitted thereto. More specifically, the photodiode PD is an organic photodiode (OPD) including an organic semiconductor. The photodiodes PD are arranged in the second direction Dy in the detection area AA.

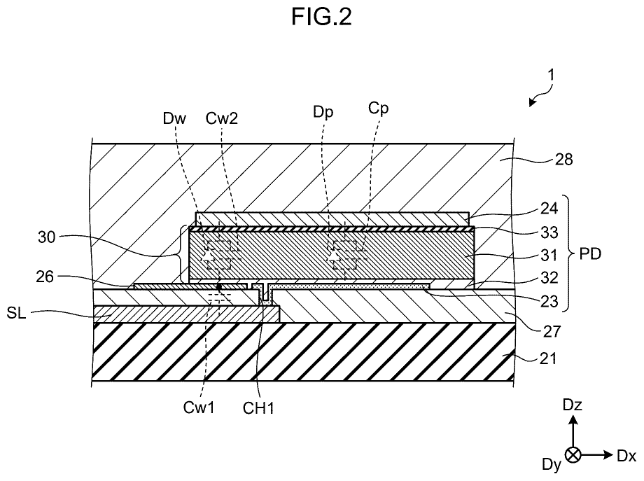

The photodiodes PD each include an organic semiconductor layer 30 (a lower buffer layer 32, an active layer 31, and an upper buffer layer 33 (refer to FIG. 2)), a lower electrode 23 disposed below the organic semiconductor layer 30, and an upper electrode 24 disposed on the upper side of the organic semiconductor layer 30. A plurality of the lower electrodes 23 are provided, one for each of the photodiodes PD, and are arranged in the second direction Dy in the detection area AA. The lower electrodes 23 are arranged apart from one another in the second direction Dy. The organic semiconductor layer 30 and the upper electrode 24 are provided across the photodiodes PD and are provided continuously in the detection area AA. To facilitate viewing of the drawing, FIG. 1 illustrates the organic semiconductor layer 30 and the upper electrode 24 provided on the upper side of the lower electrode 23 with a dashed line and a long dashed double-short dashed line, respectively. The multilayer configuration of the photodiodes PD, the lower electrodes 23, and the upper electrode 24 will be described later with reference to FIG. 2.

The signal lines SL are each electrically coupled to a corresponding one of the lower electrodes 23 of the photodiodes PD. Specifically, in the example illustrated in FIG. 1, the signal lines SL are each coupled to a corresponding one of the lower electrodes 23 through a contact hole CH1 formed in an insulating film 27 (refer to FIG. 2).

Each of the signal lines SL extends in the first direction Dx from a coupling point (contact hole CH1) with the lower electrode 23, bends to the second direction Dy, and extends in the second direction Dy along the arrangement direction of the photodiodes PD. Portions of the signal lines SL extending in the second direction Dy are arranged in the first direction Dx. The signal lines SL are coupled to a detection circuit 48 included in the control circuit 122. In other words, the detection circuit 48 is electrically coupled to the lower electrodes 23 of the photodiodes PD through the signal lines SL.

Each of the signal lines SL and each of the shield layers 26 are provided for a corresponding one of the photodiodes PD. The shield layers 26 are arranged so as to overlap the respective signal lines SL in plan view. In more detail, the shield layers 26 each overlap a portion of a corresponding one of the signal lines SL extending in the first direction Dx and extend in the first direction Dx along the signal lines SL. Each of the shield layers 26 extends across the detection area AA and the peripheral area GA. The shield layers 26 are arranged in the second direction Dy so as to overlap the respective signal lines SL.

The shield layers 26 are coupled to a power supply circuit 123 included in the control circuit 122 via the power supply wiring lines CL1 and CL2 extending in the second direction Dy. More specifically, the power supply wiring line CL1 is provided in the same layer as the shield layers 26 and is provided so as to intersect the shield layers 26. As a result, the shield layers 26 are collectively coupled to the same power supply wiring line CL1. The power supply wiring line CL2 is provided in the same layer as the signal lines SL and is electrically coupled to the power supply wiring line CL1 through a contact hole CH2. The power supply wiring line CL2 is electrically coupled to the power supply circuit 123.

With such a configuration, the power supply circuit 123 supplies a reference voltage VCOM to the shield layers 26 via the power supply wiring lines CL1 and CL2. The reference voltage VCOM is a voltage signal having a fixed predetermined potential. The reference voltage VCOM is, for example, a voltage signal having an equal potential to a reference potential REF (first reference power supply) supplied to the lower electrodes 23. The power supply wiring line CL1 is provided adjacent to the organic semiconductor layer 30 in the first direction Dx. However, the coupling between the shield layers 26 and the power supply circuit 123 may have any configuration, and the arrangement, the number, and the like of the power supply wiring lines CL1 and CL2 can be changed as appropriate.

The upper electrode 24 is provided so as to extend in the second direction Dy across the detection area AA and the peripheral area GA. That is, the upper electrode 24 is provided so as to extend from an area overlapping the organic semiconductor layer 30 to an area not overlapping the organic semiconductor layer 30, and is electrically coupled to the power supply wiring line CL3 in the area not overlapping the organic semiconductor layer 30. The power supply wiring line CL3 is provided in the same layer as the signal lines SL and is electrically coupled to the upper electrode 24 through a contact hole CH3 and a terminal 24a. The terminal 24a is provided in the same layer as the lower electrode 23.

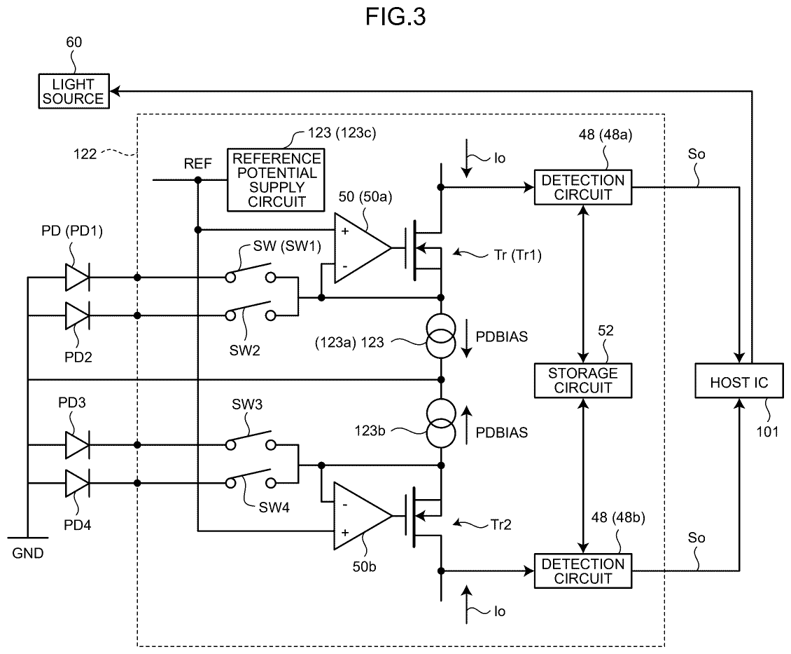

With such a configuration, the upper electrode 24 of the photodiodes PD is coupled to the power supply circuit 123 included in the control circuit 122 via the terminal 24a and the power supply wiring line CL3. The power supply circuit 123 supplies a reference potential GND (second reference power supply) (refer to FIG. 3) to the upper electrode 24 of the photodiodes PD. The reference potential GND is a predetermined fixed potential, such as a ground potential.

The control circuit 122 (detection circuit 48 and power supply circuit 123) is located adjacent to the photodiodes PD in the second direction Dy in the peripheral area GA of the substrate 21. The control circuit 122 is a circuit that controls detection operations by supplying control signals to the photodiodes PD. Each of the photodiodes PD outputs, to the detection circuit 48, the electrical signal corresponding to the light emitted thereto as a detection signal Vdet. Thereby, the detection device 1 detects information on an object to be detected based on the detection signals Vdet from the photodiodes PD.

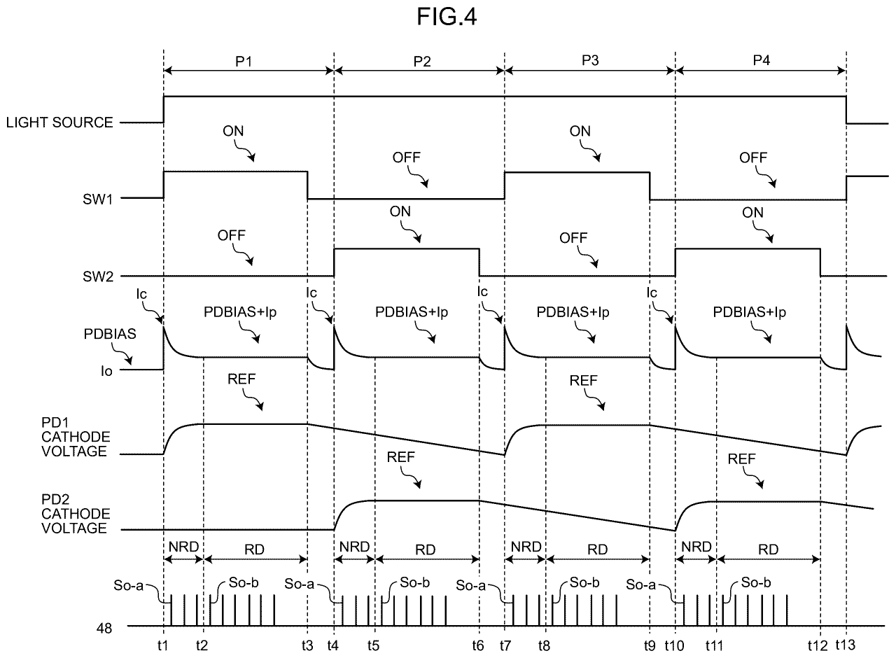

A detailed configuration of the control circuit 122 (detection circuit 48 and power supply circuit 123) and the detection operations of the photodiodes PD will be described later with reference to FIGS. 3 and 4. The control circuit 122 (detection circuit 48 and power supply circuit 123) is provided on the same substrate 21 as the photodiodes PD, but is not limited to this configuration. The control circuit 122 (detection circuit 48 and power supply circuit 123) may be provided on another control substrate coupled to the substrate 21, for example, through a flexible printed circuit board or the like. The detection circuit 48 and the power supply circuit 123 may each be formed as an individual circuit.

Although not illustrated in FIG. 1, the detection device 1 may include one or more light sources 60 (refer to FIG. 3). For example, an inorganic light-emitting diode (LED) or an organic electroluminescent (EL) diode (organic light-emitting diode (OLED)) is used as the light source 60.

Light emitted from the light source is reflected by the object to be detected such as a finger and enters the photodiodes PD. As a result, the detection device 1 can detect a fingerprint by detecting a shape of asperities on the surface of the finger or the like. Alternatively, the light emitted from the light source may be reflected in the finger or the like, or transmitted through the finger or the like, and enter the photodiodes PD. As a result, the detection device 1 can detect information on a living body in the finger or the like. Examples of the information on the living body include, but are not limited to, pulse waves, pulsation, and a vascular image of the finger or a palm. That is, the detection device 1 may be configured as a fingerprint detection device to detect a fingerprint or a vein detection device to detect a vascular pattern of, for example, veins.

The following describes a multilayer configuration of the photodiode PD and the shield layer 26. FIG. 2 is a sectional view along II-II′ of FIG. 1.

In the following description, a direction from the substrate 21 toward a sealing film 28 in a direction orthogonal to a surface of the substrate 21 is referred to as “upper side” or simply “above”. A direction from the sealing film 28 toward the substrate 21 is referred to as “lower side” or simply “below”.

As illustrated in FIG. 2, the substrate 21 is an insulating substrate and is made using, for example, glass or a resin material. The substrate 21 is not limited to having a flat plate shape and may have a curved surface. In this case, the substrate 21 may be made of a film-like resin.

The signal line SL is provided on the substrate 21. The signal line SL is formed of, for example, metal wiring, and is formed of a material having better conductivity than the lower electrode 23 of the photodiode PD. A portion of the signal line SL (the right end side of the signal line SL in FIG. 2) is provided in a layer between the substrate 21 and the photodiode PD in the third direction Dz. The insulating film 27 is provided on the substrate 21 so as to cover the signal line SL. The insulating film 27 may be an inorganic insulating film or an organic insulating film. The insulating film 27 may be a single layer or a multilayered film.

The photodiode PD is provided on the insulating film 27. In more detail, the photodiode PD includes the lower electrode 23, the lower buffer layer 32, the active layer 31, the upper buffer layer 33, and the upper electrode 24. In the photodiode PD, the lower electrode 23, the lower buffer layer 32 (electron transport layer), the active layer 31, the upper buffer layer 33 (hole transport layer), and the upper electrode 24 are stacked in this order in the direction orthogonal to the substrate 21.

The lower electrode 23 is provided on the insulating film 27 and is electrically coupled to the signal line SL through the contact hole CH1 provided in the insulating film 27. The lower electrode 23 is a cathode electrode of the photodiode PD and is formed of, for example, a light-transmitting conductive material such as indium tin oxide (ITO). The detection device 1 of the present embodiment is formed as a bottom-illuminated optical sensor in which the light from the object to be detected passes through the substrate 21 and enters the photodiode PD. The detection device 1 is, however, not limited thereto, and may be a top-illuminated optical sensor.

The active layer 31 changes in characteristics (for example, voltage-current characteristics and resistance value) depending on light emitted thereto. An organic material is used as a material of the active layer 31. Specifically, the active layer 31 has a bulk heterostructure containing a mixture of a p-type organic semiconductor and an n-type fullerene derivative ((6,6)-phenyl-Ca-butyric acid methyl ester (PCBM)) that is an n-type organic semiconductor. As the active layer 31, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 31 can be formed by a vapor deposition process (dry process) using any of the low-molecular-weight organic materials listed above. In this case, the active layer 31 may be, for example, a multilayered film of CuPc and F₁₆CuPc, or a multilayered film of rubrene and C₆₀. The active layer 31 can also be formed by a coating process (wet process). In this case, the active layer 31 is made using a material obtained by combining any of the above-listed low-molecular-weight organic materials with a high-molecular-weight organic material. As the high-molecular-weight organic material, for example, poly(3-hexylthiophene) (P3HT) and F₈-alt-benzothiadiazole (F₈BT) can be used. The active layer 31 can be a film made of a mixture of P3HT and PCBM, or a film made of a mixture of F₈BT and PDI.

The lower buffer layer 32 is an electron transport layer and the upper buffer layer 33 is a hole transport layer. The lower buffer layer 32 and the upper buffer layer 33 are provided to facilitate holes and electrons generated in the active layer 31 to reach the lower electrode 23 or the upper electrode 24. The lower buffer layer 32 is in direct contact with the top of the lower electrode 23, and is also provided in areas between the adjacent lower electrodes 23. The active layer 31 is in direct contact with the top of the lower buffer layer 32. The upper buffer layer 33 is in direct contact with the top of the active layer 31, and the upper electrode 24 is in direct contact with the top of the upper buffer layer 33.

Polyethylenimine ethoxylated (PEIE) is used as a material of the electron transport layer. The material of the hole transport layer is a metal oxide layer. For example, tungsten oxide (WO₃) or molybdenum oxide is used as the metal oxide layer.

The materials and the manufacturing methods of the lower buffer layer 32, the active layer 31, and the upper buffer layer 33 are merely exemplary, and other materials and manufacturing methods may be used. For example, each of the lower buffer layer 32 and the upper buffer layer 33 is not limited to a single-layer film, but may be formed as a multilayered film that includes an electron block layer and a hole block layer.

The upper electrode 24 is provided on the upper buffer layer 33. The upper electrode 24 is an anode electrode of the photodiode PD and is continuously formed over the entire detection area AA. In other words, the upper electrode 24 is continuously provided on the photodiodes PD. The upper electrode 24 faces the lower electrodes 23 with the lower buffer layer 32, the active layer 31, and the upper buffer layer 33 interposed therebetween. The upper electrode 24 is formed, for example, of a light-transmitting conductive material such as ITO or indium zinc oxide (IZO).

The sealing film 28 is provided on the upper electrode 24. An inorganic film, such as a silicon nitride film or an aluminum oxide film, or a resin film, such as an acrylic film, is used as the sealing film 28. The sealing film 28 is not limited to a single layer, and may be a multilayered film having two or more layers obtained by combining the inorganic film with the resin film mentioned above. The sealing film 28 well seals the photodiode PD, and thus can reduce moisture entering the photodiode PD from the upper surface side thereof.

The shield layer 26 is provided in the same layer as the lower electrode 23 on the insulating film 27. The shield layer 26 is formed of the same material as the lower electrode 23, for example, a light-transmitting conductive material such as ITO. However, the shield layer 26 is not limited to this material, and may be formed of a material different from that of the lower electrode 23, for example, a metal material.

The shield layer 26 is disposed with a gap interposed between itself and the lower electrode 23 in the first direction Dx. The shield layer 26 faces the signal line SL with the insulating film 27 interposed therebetween in the third direction Dz. A portion of the shield layer 26 is disposed between the signal line SL and the lower buffer layer 32 of the photodiode PD in the third direction Dz. In other words, the organic semiconductor layer 30 (lower buffer layer 32, active layer 31, and upper buffer layer 33) is provided so as to cover the lower electrode 23 and the portion of the shield layer 26.

The shield layers 26 are supplied with the reference voltage VCOM. As a result, the shield layer 26 reduces parasitic capacitance between the upper electrode 24 of the photodiode PD and the signal line SL, and reduces unintended capacitive coupling between the photodiode PD (upper electrode 24) and the signal line SL.

The detection device 1 of the present embodiment may have a configuration without the shield layer 26. While the example has been described where the lower electrode 23 is a cathode electrode and the upper electrode 24 is an anode electrode, the present disclosure is not limited to this example. The lower electrode 23 may be an anode electrode and the upper electrode 24 may be a cathode electrode. In that case, the lower buffer layer 32 may be a hole transport layer, and the upper buffer layer 33 may be an electron transport layer.

The following describes an exemplary detection method of the detection device 1 of the present embodiment. FIG. 3 is a circuit diagram illustrating a configuration example of the control circuit according to the embodiment. As illustrated in FIG. 3, a plurality of photodiodes PD1, PD2, PD3, and PD4 are coupled to the control circuit 122. The control circuit 122 includes a plurality of switches SW1, SW2, SW3, and SW4, operational amplifiers 50, transistors Tr, and a storage circuit 52, in addition to the detection circuit 48 and the power supply circuit 123 described above.

The anodes of the photodiodes PD1, PD2, PD3, and PD4 are supplied with the reference potential GND from the power supply circuit 123. The cathodes of the photodiodes PD1, PD2, PD3, and PD4 are coupled to the detection circuit 48 via the switches SW1, SW2, SW3, and SW4, the operational amplifiers 50, and the transistors Tr. The detection circuit 48 outputs, to a host integrated circuit (IC) 101, sensor values So corresponding to currents (photocurrents Ip) output from the photodiodes PD1, PD2, PD3, and PD4.

In more detail, the switches SW1 and SW2, an operational amplifier 50a, a transistor Tr1, and a detection circuit 48a are provided correspondingly to the two photodiodes PD1 and PD2. The photodiodes PD1 and PD2 coupled to the detection circuit 48a are switched from one to another by turning-on (coupled state) or turning-off (non-coupled state) of the switches SW1 and SW2.

In addition, the switches SW3 and SW4, an operational amplifier 50b, a transistor Tr2, and a detection circuit 48b are provided correspondingly to the two photodiodes PD3 and PD4. The photodiodes PD3 and PD4 coupled to the detection circuit 48b are switched from one to another by turning-on or turning-off of the switches SW3 and SW4. The power supply circuit 123 includes constant current sources 123a and 123b and a reference potential supply circuit 123c.

In the following description, a circuit configuration corresponding to the photodiodes PD1 and PD2 will be described. The circuit configuration corresponding to the photodiodes PD1 and PD2 is the same as a circuit configuration corresponding to the photodiodes PD3 and PD4. The description of the circuit configuration corresponding to the photodiodes PD1 and PD2 is applicable to the circuit configuration corresponding to the photodiodes PD3 and PD4.

In the following description, the transistors Tr1 and Tr2 will each be simply referred to as a “transistor Tr” when need not be distinguished from each other. The detection circuits 48a and 48b will each be simply referred to as a “detection circuit 48” when need not be distinguished from each other. The operational amplifiers 50a and 50b will each be simply referred to as an “operational amplifier 50” when need not be distinguished from each other.

As illustrated in FIG. 3, the operational amplifier 50 and the transistor Tr are coupled between the switches SW1, SW2 coupled to the photodiodes PD1, PD2 and the detection circuit 48.

One end of the switch SW1 and one end of the switch SW2 are coupled to the cathode of the photodiode PD1 and the cathode of the photodiode PD2, respectively. The other ends of the switches SW1, SW2 are coupled to the inverting input terminal (−) of the operational amplifier 50 and the source of the transistor Tr. That is, the inverting input terminal (−) of the operational amplifier 50 is coupled to the photodiodes PD1, PD2 via the switches SW1, SW2. The non-inverting input terminal (+) of the operational amplifier 50a is supplied with the reference potential REF from the reference potential supply circuit 123c. The output of the operational amplifier 50 is coupled to the gate of the transistor Tr.

The transistor Tr is a source follower transistor. The source of the transistor Tr is coupled to the inverting input terminal (−) of the operational amplifier 50, and also coupled to the reference potential GND via the constant current source 123a. The drain of the transistor Tr is coupled to the detection circuit 48.

The detection circuit 48 is a current detection circuit that measures a current flowing through the drain of the transistor Tr. The detection circuit 48 is configured, for example, with an analog-to-digital (A/D) conversion circuit, a signal processing circuit, and the like. The detection circuit 48 outputs a sensor value So by performing signal processing such as the A/D conversion based on the current flowing through the drain of the transistor Tr. While FIG. 3 is illustrated in a simplified manner, the current may be measured by providing a resistor so as to be coupled on the drain side of the transistor Tr and measuring a voltage between opposite ends of the resistor, or may be measured by using other methods.

The storage circuit 52 stores therein information on detection conditions for the detection circuit 48. Specifically, the storage circuit 52 stores therein information on a non-readout period NRD and a readout period RD that have been set in advance for each of detection periods P1, P2, P3, and P4 (refer to FIG. 4). The storage circuit 52 alternatively stores therein information on the number of times of measurement of the current flowing through the drain of the transistor Tr for each of the detection periods P1, P2, P3, and P4 (refer to FIG. 4). The information on the number of times of measurement includes also information on the number of times of measurement in the non-readout period NRD and the number of times of measurement in the readout period RD. The storage circuit 52 is a register circuit, for example. With such a configuration, when, for example, the switch SW1 is turned on and the switch SW2 is turned off, the photodiode PD1 is coupled to the detection circuit 48 via the switch SW1, the operational amplifier 50, and the transistor Tr. The photodiode PD2 is decoupled from the detection circuit 48. Due to a virtual short circuit of the operational amplifier 50, the potential of the cathode of the photodiode PD1 becomes the reference potential REF that is the same as the potential of the non-inverting input terminal (+).

The reference potential REF is higher than the reference potential GND. As a result, the photodiode PD1 is driven in a reverse-biased manner when the switch SW1 is turned on. A bias current PDBIAS from the constant current source 123a and a photocurrent Ip corresponding to the light emitted to the photodiode PD1 flow through the drain of the transistor Tr. The detection circuit 48 measures the photocurrent Ip output from the photodiode PD1, and outputs the sensor value So corresponding to the photocurrent Ip. Since an output current Io (to be described later) flowing through the drain of the transistor Tr is given by Io=PDBIAS+Ip, the photocurrent Ip is given by Ip=Io−PDBIAS. The bias current PDBIAS is a preset current.

In the same way, when the switch SW1 is turned off and the switch SW2 is turned on, the photodiode PD2 is coupled to the detection circuit 48 via the switch SW2, the operational amplifier 50, and the transistor Tr. The photodiode PD1 is decoupled from the detection circuit 48. Due to the virtual short circuit of the operational amplifier 50, the potential of the cathode of the photodiode PD2 becomes the reference potential REF that is the same as the potential of the non-inverting input terminal (+). As a result, the photodiode PD2 is driven in a reverse-biased manner. The bias current PDBIAS from the constant current source 123a and the photocurrent Ip corresponding to the light emitted to the photodiode PD2 flow through the drain of the transistor Tr. The detection circuit 48 measures the photocurrent Ip output from the photodiode PD2, and outputs the sensor value So corresponding to the photocurrent Ip.

With the circuit configuration illustrated in FIG. 3, the photodiodes PD1, PD2 coupled to the operational amplifier 50, the transistor Tr, and the detection circuit 48 are switched from one to another in a time-division manner by turning-on and turning-off of the switches SW1 and SW2. Therefore, the detection device 1 can reduce the circuit size compared with a case where the operational amplifier 50, the transistor Tr, and the detection circuit 48 are provided corresponding to each of the photodiodes PD1 and PD2. The detection device 1 can also increase the degree of flexibility of the detection; for example, the detection device 1 can perform the detection using light having different wavelengths or detect different objects to be detected, correspondingly to the photodiodes PD1 and PD2.

FIG. 4 is a timing waveform diagram illustrating an operation example of the detection device according to the embodiment. As illustrated in FIG. 4, the detection device 1 has the detection periods P1, P2, P3, and P4. In the detection periods P1 and P3, the detection circuit 48 detects the photocurrent Ip corresponding to the light emitted to the photodiode PD1. In the detection periods P2 and P4, the detection circuit 48 detects the photocurrent Ip corresponding to the light emitted to the photodiode PD2. In the following description, the detection periods P1, P2, P3, and P4 may each be simply referred to as a “detection period P” when need not be distinguished from one another.

In more detail, the light source 60 is continuously on over the detection periods P1, P2, P3, and P4 based on a control signal from the host IC 101. The switches SW1 and SW2 are alternately turned on and off. In the detection period P1, the switch SW1 is on and the switch SW2 is off. In the detection period P2, the switch SW1 is off and the switch SW2 is on. In the detection period P3, the switch SW1 is on and the switch SW2 is off. In the detection period P4, the switch SW1 is off and the switch SW2 is on.

At time t1 in the detection period P1, the switch SW1 is turned on, and the photodiode PD1 is coupled to the detection circuit 48 via the switch SW1, the operational amplifier 50, and the transistor Tr. During the detection period P1, the switch SW2 is off, and the photodiode PD2 is decoupled from the detection circuit 48.

When the switch SW1 is turned on at time t1, the photodiode PD1 is driven in a reverse-biased manner, and an inrush current Ic flows into the photodiode PD1. The inrush current Ic that flows into the photodiode PD1 decreases over time; and then, from time t2, which is a predetermined period after time t1, the photocurrent Ip corresponding to the light emitted to the photodiode PD1 flows into the photodiode PD1. In the following description, a steady state denotes a state corresponding to a period starting after the current value of the inrush current Ic decreases after a lapse of the predetermined period from time t1 (period from time t2 until the switch SW1 is turned off at time t3). The inrush current Ic is a current that charges the capacitance of the photodiode PD during a period until the cathode voltage reaches the reference potential REF.

The output current Io flowing through the drain of the transistor Tr is the sum of the bias current PDBIAS and the current that flows into the photodiode PD1. That is, at time t1, the output current Io is the sum of the bias current PDBIAS and the inrush current Ic. From time t2, which is the predetermined period after time t1, the output current Io is placed in the steady state in which variations in the current value are reduced. This steady state changes with the intensity of the emitted light. During the period from time t2 until the switch SW1 is off at time t3, the output current Io is the sum of the bias current PDBIAS and the photocurrent Ip from the photodiode PD1.

When the switch SW1 is turned on at time t1, the cathode voltage of the photodiode PD1 rises to the reference potential REF. From time t2 to time t3, the cathode voltage of the photodiode PD1 is constant at the reference potential REF. During the detection period P1, the cathode voltage of the photodiode PD2 is reset to have the same potential as the reference potential GND, but is not limited to this configuration.

The detection circuit 48 measures the photocurrent Ip from the photodiode PD1 a plurality of times in a time-division manner during the detection period P1 and outputs the sensor value So corresponding to the photocurrent Ip. As illustrated in FIG. 4, the detection circuit 48 measures the photocurrent Ip output from the photodiode PD1 a preset number of times (first number of times, such as eight times) during a period from time t1 (first time point) at which the switch SW1 is turned on to time t3 (second time point) at which the switch SW1 is turned off. When the switch SW1 is turned off, the electric charge stored in the capacitance of the photodiode PD1 is discharged by the photocurrent Ip flowing into the photodiode PD1, and the cathode potential of the photodiode PD1 gradually decreases.

The detection circuit 48 has the non-readout period NRD and the readout period RD in one detection period P. The non-readout period NRD is a period from time t1, at which the switch SW1 is turned on, to time t2, that is, a period in which the inrush current Ic flows into the photodiode PD1 and the output current Io is in a non-steady state. The readout period RD is a period from time t2 to time t3 at which the switch SW1 is turned off, that is, the period in which the inrush current Ic flowing into the photodiode PD1 is smaller and the output current Io is in the steady state.

The detection circuit 48 does not output a sensor value So-a during the non-readout period NRD from time t1 (first time point), at which the switch SW1 is turned on, to time t2. Specifically, the detection circuit 48 measures the photocurrent Ip output from the photodiode PD1 a preset number of times (second number of times, such as three times) during the non-readout period NRD. However, the detection circuit 48 does not output the sensor value So-a during the non-readout period NRD. That is, since the sensor value So-a has a large error due to the effect of the inrush current Ic, the detection device 1 does not use the sensor value So-a as biometric information.

The detection circuit 48 measures the photocurrent Ip and outputs a sensor value So-b corresponding to the photocurrent Ip to the host IC 101 during the readout period RD after a lapse of a predetermined period from the time t1 (first time point) at which the switch SW1 is turned on. Specifically, the detection circuit 48 measures the photocurrent Ip output from the photodiode PD1 a preset number of times (third number of times, such as five times) during the readout period RD. The detection circuit 48 performs signal processing based on the current value acquired for each number of times of measurement in the readout period RD and outputs the sensor value So-b. The detection circuit 48 may output, as the sensor value So, the sum of the sensor values So-b measured in the readout period RD or the average of the sensor values So-b measured in the readout period RD. The detection device 1 acquires the biometric information based on sensor values So.

In other words, the detection circuit 48 measures the photocurrent Ip output from the photodiode PD1 at a predetermined cycle in a time-division manner the preset number of times (first number of times, such as eight times) during the period from time t1 (first time point) at which the switch SW1 is turned on to time t3 (second time point) at which the switch SW1 is turned off. The detection circuit 48 outputs the sensor value So-b based on the number of times (third number of times, such as five times) of measurement in the readout period RD obtained by excluding the number of times of measurement in the non-readout period NRD (second number of times at the first round, such as three times) from the preset number of times of measurement. The sensor values So output from the detection circuit 48 to the host IC 101 do not include the sensor values So-a in the non-readout period NRD and includes the sensor values So-b in the readout period RD. The storage circuit 52 stores therein in advance information on the numbers of times (first number of times, second number of times, and third number of times) of measurement of the photocurrent Ip by the detection circuit 48 and information on the lengths of the non-readout period NRD and the readout period RD.

Then, the switch SW1 is turned off at time t3, and the switch SW2 is turned on at time t4, which is a predetermined period after time t3. During the detection period P2 from time t4 to time t7, the photodiode PD2 performs detection. The detection operation of the photodiode PD2 is the same as that in the detection period P1 described above, and will not be described again.

With the configuration described above, the detection device 1 does not output the sensor value So-a during the period (from time t1 to time t2) when the inrush current Ic flowing into the photodiode PD1 has a larger effect, and outputs the sensor value So (sensor value So-b) in the steady state after the inrush current Ic flowing into the photodiode PD1 decreases. This configuration allows the detection device 1 to reduce errors in the sensor value So due to the inrush current Ic flowing into the photodiode PD1, thereby capable of improving the detection accuracy.

The circuit configuration illustrated in FIG. 3 and the timing waveform diagram illustrated in FIG. 4 are merely exemplary and can be changed as appropriate. For example, FIG. 3 illustrates the four photodiodes PD1, PD2, PD3, and PD4, but the number of the photodiodes PD is not limited to four. One to three photodiodes PD may be provided, or five or more photodiodes PD may be provided and sequentially driven in the same manner. The number of the switches SW can also be changed correspondingly to the number of the photodiodes PD.

In FIG. 4, the light source 60 is controlled to be on continuously over the detection periods P1, P2, P3, and P4, but is not limited to being controlled in this way. The light source 60 only needs to be controlled to be on at least during the readout period RD. In more detail, the light source 60 is controlled to be on at least during a period when the detection circuit 48 outputs the sensor values So-b in the period from time t1 (first time point) at which the switch SW1 is turned on to time t3 (second time point) at which the switch SW1 is turned off.

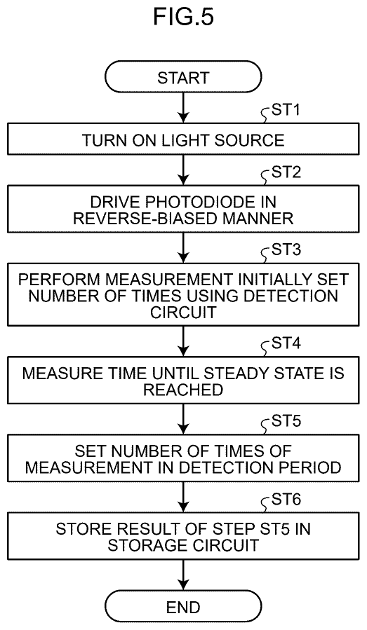

The following describes an exemplary method for setting the numbers of times (first number of times, second number of times, and third number of times) of measurement of the detection circuit 48. FIG. 5 is a flowchart illustrating an exemplary method for setting the number of times of measurement of the detection circuit of the detection device according to the embodiment.

As illustrated in FIG. 5, the light source 60 is turned on based on the control signal from the host IC 101 (Step ST1).

The switch SW1 is turned on, and the cathode of the photodiode PD1 is coupled to the inverting input terminal (−) of the operational amplifier 50 via the switch SW1. As a result, the photodiode PD1 is driven in a reverse-biased manner (Step ST2).

The detection circuit 48 measures the photocurrent Ip output from the photodiode PD1 an initially set number of times (for example, eight times) set in advance (Step ST3).

The detection circuit 48 outputs the sensor value So corresponding to the photocurrent Ip for each number of times of measurement. The host IC 101 measures the relation between the number of times of measurement (elapsed time from time t1 at which the switch SW1 is turned on) and the sensor value So (voltage), based on the sensor value So for each number of times of measurement. Then, the host IC 101 measures the time elapsed after the switch SW1 is turned on until the steady state is reached (Step ST4).

The host IC 101 sets the numbers of times (first number of times, second number of times, and third number of times) of measurement of the detection circuit 48 based on the time until the steady state is reached, which is obtained at Step ST4 (Step ST5). The host IC 101 sets the initially set number of times as the number of times (first number of times) of measurement, for example. The host IC 101 sets the number of times (second number of times at the first round) of measurement during the non-readout period NRD in the number of times (first number of times) of measurement, based on the time until the steady state is reached. The host IC 101 also sets the number of times (third number of times) of measurement during the readout period RD, by excluding the number of times (second number of times at the first round) of measurement during the non-readout period NRD from the number of times (first number of times) of measurement. Alternatively, the host IC 101 may set the number of times (third number of times) of measurement during the readout period RD, based on the magnitude (voltage value) of the sensor value So and the required detection characteristics, and set the number of times (first number of times) of measurement together with the number of times (second number of times at the first round) of measurement during non-readout period NRD, which is set based on the time until the steady state is reached.

The numbers of times (first number of times, second number of times, and third number of times) of measurement of the detection circuit 48 are stored in the storage circuit 52 (Step ST6).

The process of setting the number of times of measurement illustrated in FIG. 5 may be performed at any timing. The process may be performed at shipment of the detection device 1, or may be performed at power-on of the detection device 1 or at the start of the measurement.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiment and the modifications thereof described above.

What is claimed is:

1. A detection device comprising:
a photodiode; and
a detection circuit that is coupled to an anode or a cathode of the photodiode via a switch and is configured to output a sensor value corresponding to a current output from the photodiode, wherein
the detection circuit is configured to measure the current output from the photodiode and output the sensor value, after a lapse of a predetermined period from a first time point at which the switch is turned on.

2. The detection device according to claim 1, wherein the detection circuit is configured to measure the current output from the photodiode in a time-division manner a first number of times during a period from the first time point at which the switch is turned on to a second time point at which the switch is turned off, and output the sensor value a third number of times obtained by excluding a second number of times at a first round from the first number of times.

3. The detection device according to claim 1, comprising an operational amplifier and a source follower transistor that are coupled between the switch and the detection circuit, wherein
a source of the source follower transistor is coupled to an inverting input terminal of the operational amplifier and is coupled to a reference potential via a constant current source,
a drain of the source follower transistor is coupled to the detection circuit, and
the inverting input terminal of the operational amplifier is coupled to the photodiode via the switch.

4. The detection device according to claim 1, comprising a plurality of the photodiodes and a plurality of the switches, wherein
the photodiodes coupled to the detection circuit are switched from one to another by operation of the switches.

5. The detection device according to claim 1, comprising a light source, wherein
the light source is configured to be on at least during a period in which the detection circuit outputs the sensor value within a period from the first time point at which the switch is turned on to a second time point at which the switch is turned off.

6. The detection device according to claim 1, comprising a storage circuit configured to store information on the predetermined period.

7. The detection device according to claim 2, comprising a storage circuit configured to store the second number of times.

8. The detection device according to claim 1, wherein the photodiode is an organic photodiode (OPD).
